SEQUENCE LISTING (1 sequence)

SEQ ID NO: 1 - DNA, 456 bp, Homo sapiens
gatgatgacactattgctaaggtcaacgctgcaatgaatggacagttgccgtttgctgttgtgggaagtatggatgaggtaaaagtcggaaacaagatggtcaaagctcgccagtacccttggggtgttgtacaagtggaaaatgaaaaccactgtgactttgtaaagctgcgggaaatgctcatttgtacaaatatggaggacctgcgagagcagacccataccaggcactatgagctttacaggcgctgcaaactggaggaaatgggctttacagatgtgggcccagaaaacaagccagtcaggtagggtggggttcttgggaagccaaccaaagaggaagttagagaggattgcatatttttatatatccaggatgatggggcatgtgttctttagaatctcccaaaaccatgatatagaatcatttggtaatgactaattattgtgcttctt

(19) United States
(12) Patent Application Publication
(10) Pub. No.: US 2004/0161775 A1
(43) Pub. Date: Aug. 19, 2004

(54) Gene SHINC-1 and diagnostic and therapeutic uses thereof

(75) Inventors: Usha Kasid, Rockville, MD (US); Isamu Sakabe, Washington, DC (US); Simeng Suy, Richmond, VA (US); Deepak Kumar, Arlington, VA (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignees: Georgetown University, Washington, DC (US); NeoPharm, Inc., Lake Forest, IL

(21) Appl. No.: 10/680,313

(22) Filed: Oct. 6, 2003

Related U.S. Application Data
(63) Continuation of application No. PCT/US02/10849, filed on Apr. 8, 2002.
(60) Provisional application No. 60/281,779, filed on Apr. 6, 2001.

(51) Int. Cl. C12Q 1/68; G01N 33/574; C07H 21/04

(52) U.S. Cl. 435/6; 435/7.23; 435/69.1; 435/320.1; 435/325; 530/350; 536/23.5

(57) ABSTRACT

The partial DNA sequence of new gene SHINC-1 is provided. This gene and corresponding polypeptide have diagnostic and therapeutic application for detecting and treating cancers that involve expression of SHINC-1 such as breast cancer, leukemia, lymphoma, melanoma, colorectal cancer, and lung cancer.

### CROSS REFERENCE RELATED APPLICATIONS

This application claims benefit of priority to Provisional Application Serial No. 60/281,779, filed Apr. 6, 2001, which is incorporated by reference in its entirety herein.

### FIELD OF THE INVENTION

The present invention relates to a gene that encodes a polypeptide involved in apoptosis. This polypeptide is a useful target for identifying compounds that modulate cancer progression by modulating apoptosis. Also, this polypeptide is useful as a diagnostic target for detecting cancers wherein the expression of this polypeptide varies from its expression levels in non-cancerous cells.

### BACKGROUND OF THE INVENTION

Malignant tumors develop through a series of stepwise, progressive changes that lead to the loss of growth control characteristic of cancer cells, i.e., continuous unregulated proliferation, the ability to invade surrounding tissues, and the ability to metastasize to different organ sites. Carefully controlled in vitro studies have helped define the factors that characterize the growth of normal and neoplastic cells and have led to the identification of specific proteins that control cell growth and differentiation.

Oligonucleotides have been employed as therapeutic moieties in the treatment of disease states in animals and man. For example, workers in the field have now identified antisense, triplex and other oligonucleotide compositions which are capable of modulating expression of genes implicated in viral, fungal and metabolic diseases. tisense oligonucleotide inhibition of gene expression has proven to be a useful tool in understanding the roles of raf genes. An antisense oligonucleotide complementary to the first six codons of human c-raf has been used to demonstrate that the mitogenic response of T cells to interleukin-2 (IL-2) requires c-raf. Cells treated with the oligonucleotide showed a near-total loss of c-raf protein and a substantial reduction in proliferative response to IL-2. Riedel et al., Eur. J. Immunol. 1993, 23, 3146-3150. Rapp et al. have disclosed expression vectors containing a raf gene in an antisense orientation downstream of a promoter, and methods of inhibiting raf expression by expressing an antisense Raf gene or a mutated Raf gene in a cell. WO application 93/04170. An antisense oligodeoxyribonucleotide complementary to codons 1-6 of murine c-Raf has been used to abolish insulin stimulation of DNA synthesis in the rat hepatoma cell line H4IIE. Tornkvist et al., J. Biol. Chem. 1994, 269, 13919-13921. WO Application 93/06248 discloses methods for identifying an individual at increased risk of developing cancer and for determining a prognosis and proper treatment of patients afflicted with cancer comprising amplifying a region of the c-raf gene and analyzing it for evidence of mutation. Denner et al. discloses antisense polynucleotides hybridizing to the gene for raf, and processes using them. WO 94/15645. Oligonucleotides hybridizing to human and rat raf sequences are disclosed. Iversen et al. discloses heterotypic antisense oligonucleotides complementary to raf which are able to kill ras-activated cancer cells, and methods of killing raf-activated cancer cells. Numerous oligonucleotide sequences are disclosed, none of which are actually antisense oligonucleotide sequences.

Other approaches to the therapeutic control of the proliferation and death of cancerous cells involve small molecular weight chemical agents that play a role in modulating apoptosis. One such molecule is Tempo. The present inventors have recently shown that tempo, a low molecular weight antioxidant, is a novel inducer of apoptosis (Suy et al., JBC, 273:17871, 1998, and International Application No. PCT/US99/14173; the contents of which are hereby incorporated by reference in their entirety). Tempo-treatment of tumor-bearing athymic mice causes tumor growth arrest or tumor regression. It is therefore desirable to identify genes the expression of which may be modulated by exposition to Tempo. The identification of such genes is highly beneficial in designing novel gene-based cancer therapeutic and diagnostic protocols.

### OBJECTS AND SUMMARY OF THE INVENTION

It is an object of the invention to provide a novel gene that encodes a polypeptide the expression of which is modulated by exposing a cell to Tempo.

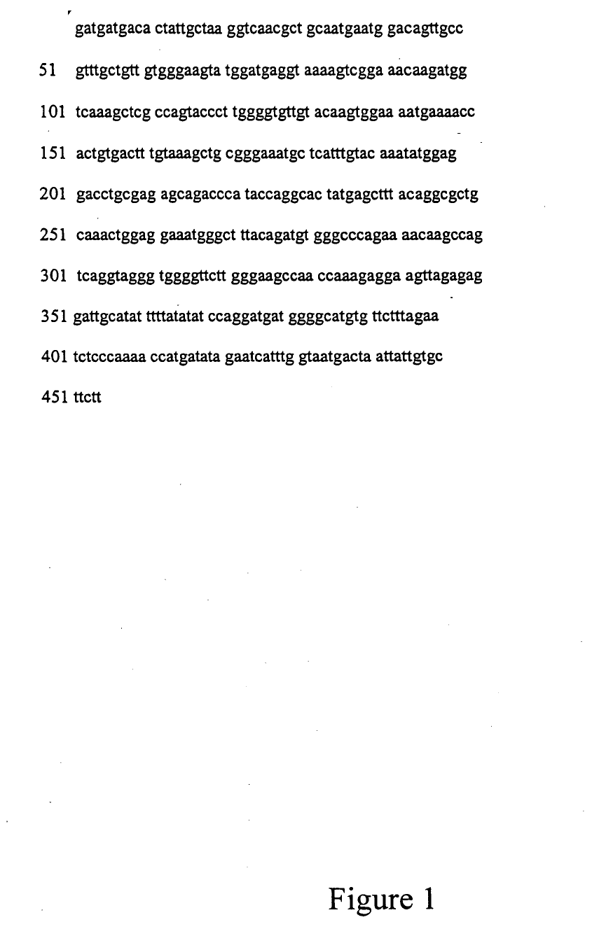

It is an object of the invention to provide a gene that corresponds to a nucleic acid sequence comprising the nucleic acid sequence set forth in FIG. 1 (SEQ ID NO 1).

It is a more specific object of the invention to provide a SHINC-1 nucleic acid sequence identified in FIG. 1 having SEQ ID NO 1.

It is another specific object of the invention to provide a nucleic acid sequence corresponding to nucleotides 1 to 485 of SEQ ID NO 1 contained in FIG. 1 or a fragment thereof which is at least 100 nucleotides in length.

It is another object of the invention to provide a SHINC-1 polypeptide that modulated apoptosis comprising an amino acid sequence which sequence is encoded by the nucleic acid sequence depicted in FIG. 1, or a fragment thereof which is at least 50 amino acids in length or an analog or homolog having at least 90% sequence identity to said polypeptide which modulates apoptosis.

It is another object of the invention to provide an antibody that specifically binds SHINC-1 polypeptide.

It is another specific object of the invention to provide a method for identifying compounds that modulate apoptosis by screening for compounds that specifically bind SHINC-1 polypeptide.

It is another specific object of the invention to provide a method for detecting or evaluating the prognosis of a cancer characterized by a change in expression of SHINC-1 compared to expression levels thereof in normal cells by detecting expression of SHINC-1 in an analyte obtained from a patient tested for cancer and correlating the level of expression to a positive or negative diagnosis for cancer.

It is another object to provide a method of treating or preventing a cancer characterized by variation in the expression of SHINC-1 comprising administering a compound that inhibits or promotes SHINC-1 gene expression and/or activity of SHINC-1 polypeptide.

It is yet another object to provide a method for treating cancer comprising administering at least one antisense oligonucleotide or ribozyme that inhibits SHINC-1 expression, thereby modulating cancer cell proliferation and/or metastatic potential.

It is still another object of the invention to provide a pharmaceutical composition for treatment of cancer that comprises an agonist or an antagonist of SHINC-1 expression and/or activity and a pharmaceutically acceptable carrier. Preferably, such compositions will comprise liposomal formulations.

Another object of the invention is to provide diagnostic compositions for detection of cancer that comprise an oligonucleotide that specifically binds at least five nucleotides of SHINC-1 DNA or an antibody that specifically binds the SHINC-1 polypeptide, attached directly or indirectly to a label, and a diagnostically acceptable carrier.

It is another object of the invention to provide methods for inhibiting tumor growth and/or metastasis by administration of a molecule that modulates the expression and/or activity of SHINC-1.

It is a preferred object of the invention to provide liposomal formulations for antisense therapy that modulate tumor growth and/or metastasis which comprise antisense oligonucleotides specific to SHINC-1, optionally in association with cytotoxic moieties such as radionuclides.

### DETAILED DESCRIPTION OF THE FIGURES

FIG. 1. Partial cDNA sequence of Shinc-1 gene. The partial nucleotide sequence of a Shinc-1 cDNA fragment (456 bp) isolated from human prostate cancer cells (DU-145) by the differential display of mRNA approach is shown (GenBank Accession no. AF316880). The nucleotide sequence representing the anchor primer and the arbitrary primer was confirmed as flanking sequence (data not shown). Based on the genomic database search, Shinc-1 gene is located on chromosome 8 (GenBank Accession no. AC084709, clone RP11-988M20).

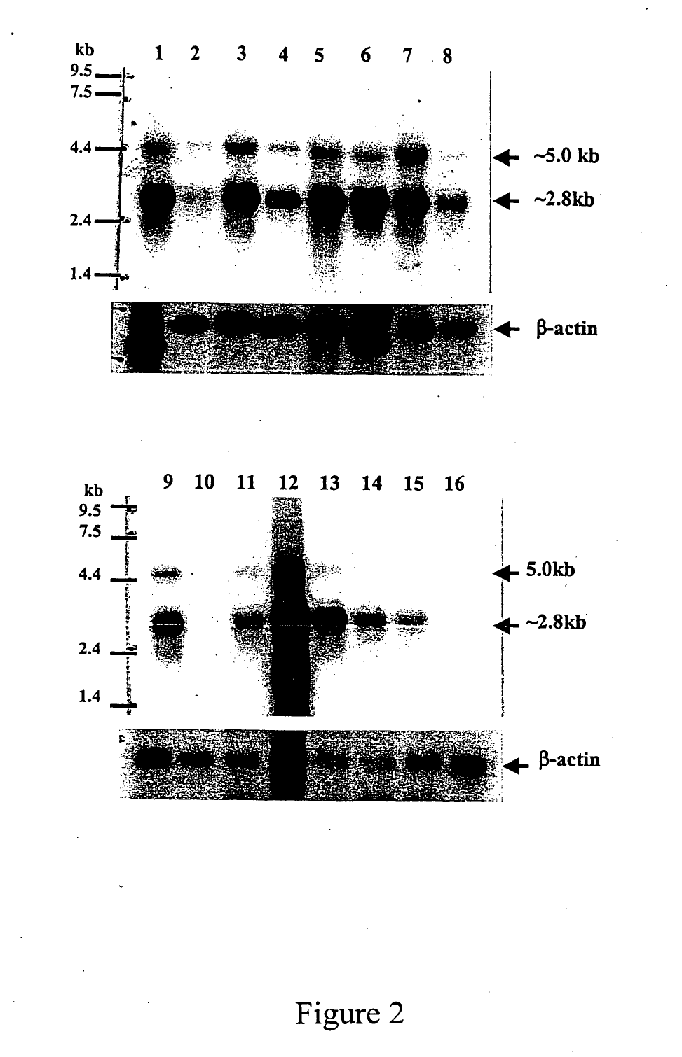

FIG. 2. Expression on Shinc-1 mRNA in normal human tissues. Blots were hybridized with radiolabeled Shinc-1 cDNA probe followed by β-actin cDNA probe. Lane 1: Heart; lane 2: Brain; lane 3: Placenta; lane 4: Lung; lane 5: Liver; lane 6: Skeltal muscle; lane 7: Kidney; lane 8: Pancreas; lane 9: Spleen; lane 10: Thymus; lane 11: Prostate; lane 12: Testis; lane 13: Ovary; lane 14: Small intestine; lane 15: Colon; lane 16: Peripheral blood leukocytes. Approximately 5.0 kb and 2.8 kB Shinc-1 transcripts are shown.

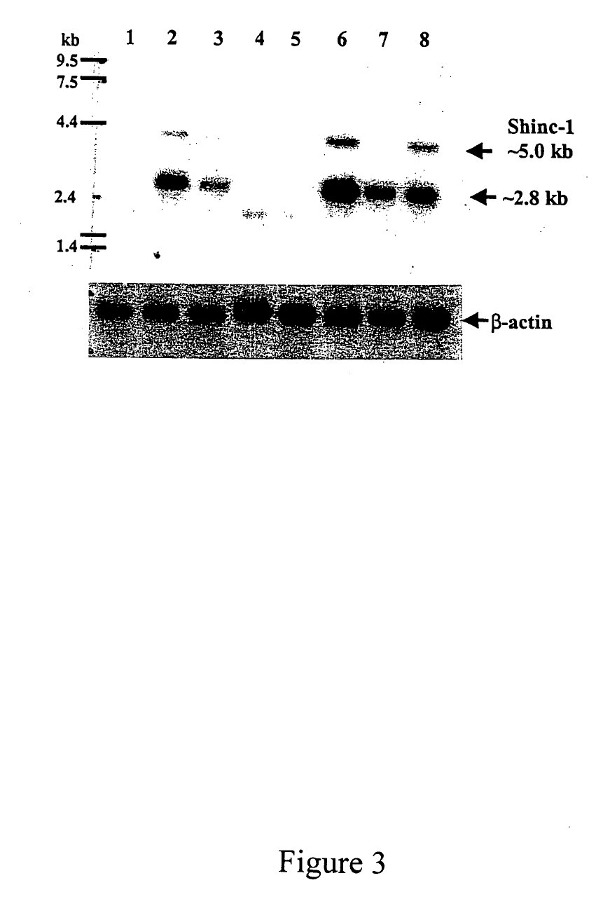

FIG. 3. Expression of Shinc-1 mRNA in human cancer cells. Blots were sequentially probed with radiolabeled Shinc-1 (upper panels) and β-actin (lower panels) cDNA probes. HL-60, promyelocytic leukemia (lane 1); HeLa-S3, (lane 2); K-562, chronic myelogenous leukemia (lane 3); MOLT-4, lymphoblastic leukemia (lane 4); BL-RAJI, Burkitt's lymphoma (lane 5); SW480, colorectal adenocarcinoma (lane 6); A549, lung carcinoma (lane 7); G361, melanoma (lane 8).

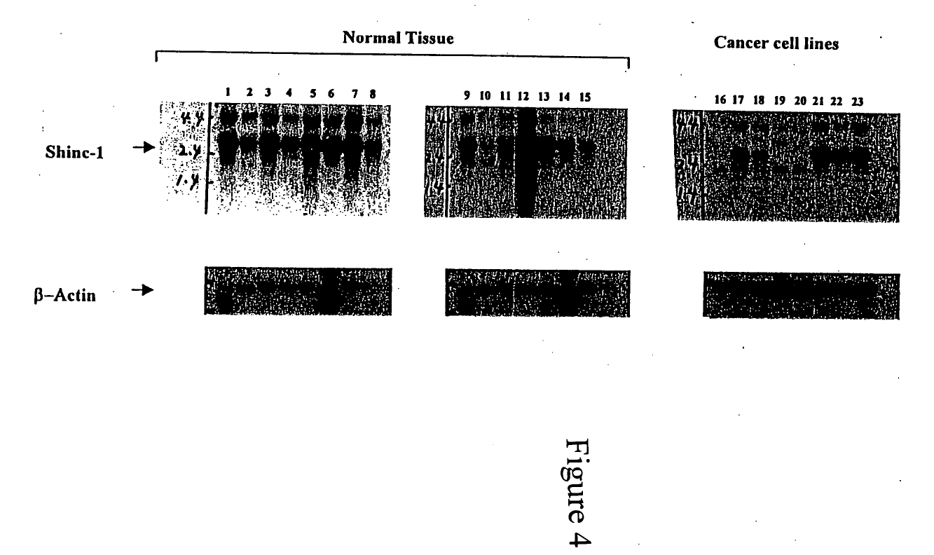

FIG. 4. The mRNA expression (of Shinc-1 gene in normal human tissues and human cancer cell lines. Human adult normal tissue and cancer cell-lines blots (Clontech) were probed with a radiolabeled partial Shinc-1 cDNA fragment. The blots were reprobed with beta-actin. Autoradiographs were scanned using Image Quant program (Molecular Dynamics, Inc.) and Shinc-1 expression was normalized to beta actin in corresponding lane. Lanes 1, Heart; 2, Brain; 3, Placenta; 4, Lung; 5, Liver; 6, Skeletal muscle; 7, Kidney; 8, Pancreas; 9, Spleen; 10, Thymus; 11, Prostate; 12, Testis; 13, Ovary; 14, Small intestine; 15, Colon; 16, Peripheral blood leukocytes; 17, Promyelocytic leukemia (HL-60); 18, Hela S3; 19, Cronic myleogenous leukemia (K-562); 20, Lymphoblastic leukemia (MOLT-4); 21, Burkitt's lymphoma Raji; 22, Colorectal adenocarcinoma (SW-480); 22, Lung carcinoma (A-549); 23, Melanoma (G-361).

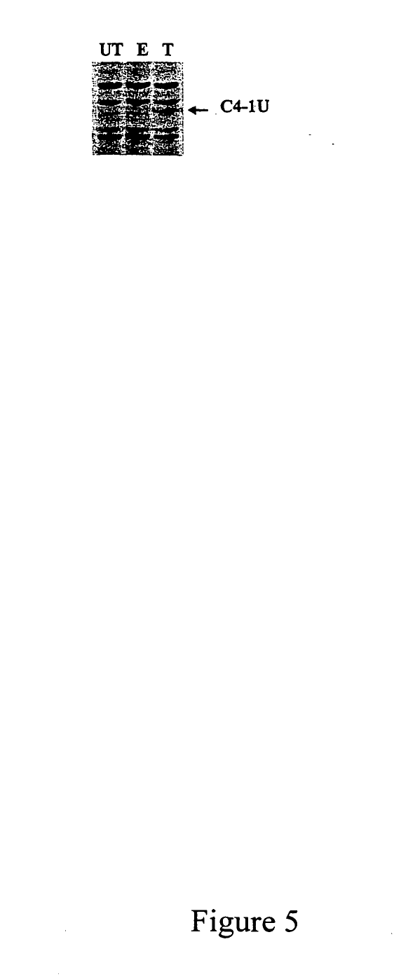

FIG. 5. Identification of differentially expressed C4-1U cDNA fragment in DU-145 cells treated with tempo. DU-145 cells were treated for 2 h with 7.5 mM tempo (T), or vehicle (ethanol 1%. E) or left untreated (UT). Total cellular RNA was extracted using RNAzol B (Tel-Test Inc, Texas). The RNA was then further cleaned off chromosomal contamination by treating with DNase 1 using the MessageClean kit according to the manufacturer's instructions (GenHunter, Brookline, Mass.). 0.2 μg of RNA was used in reverse transcription (RT) reaction in the presence of 0.2 μM of anchor primer and 100 U of MMLV Reverse Transcriptase (GenHunter), according to the manufacturer's instructions (GenHunter). cDNA was then either stored at −20° C. or used in a polymerase chain reaction (PCR). PCR was carried out with the RNAimage kit (GenHunter) according to the manufacturer's instructions. Briefly 2 μl of RT mix was used in a reaction with 0.2 μM of the same anchor primer as used in the cDNA generation, 0.2 μM of an arbitrary primer, 2 μM dNTP, 10 μCi ³³P-dATP (1250 Ci/mmol; NEN Dupont, Boston, Mass.) and 1 unit of Amplitaq (Perkin Elmer, Branchburg, N.J.). The reactions were subjected to 40 cycles at 94° C. for 30 sec, 40° C. for 2 min, and 72° C. for 30 sec, followed by a final soak temperature of 72° C. for 5 min on the 9600 Perkin Elmer thermal cycler (Perkin Elmer). The reactions were then stored at −20° C. To examine. the differentially displayed mRNAs, 3.5 μl of sample was mixed with 2 μl of loading dye (GenHunter), incubated at 80° C. for 5 min, and electrophoresed on a 6% denaturing polyacrylamide gel, followed by autoradiography.

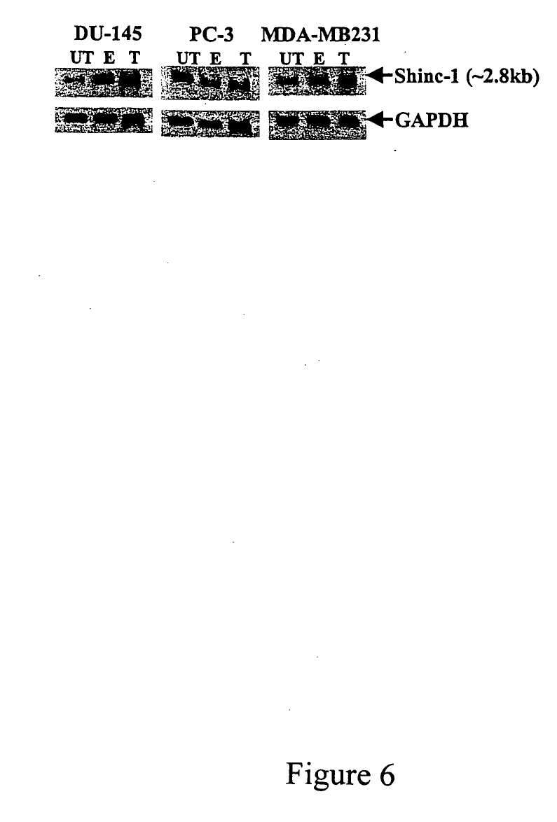

FIG. 6. Northern blot hybridization analysis of Shinc-1 gene expression in tempo-treated human prostate (DU-145 and PC-3) and breast cancer cells (MDA-MB 231). Total RNA was extracted from the cells untreated, treated with 1% ethanol or 7.5 mM tempo for 2 h. The RNA was subjected to electrophoresis in 1% formaldehyde agarose gels and transferred onto nylon membranes. Northern blots were sequentially hybridized first with a radiolabeled partial human Shinc-1 cDNA probe, followed by the radiolabeled GAPDH cDNA probe. UT, untreated; E, ethanol treated; T, tempo treated cells

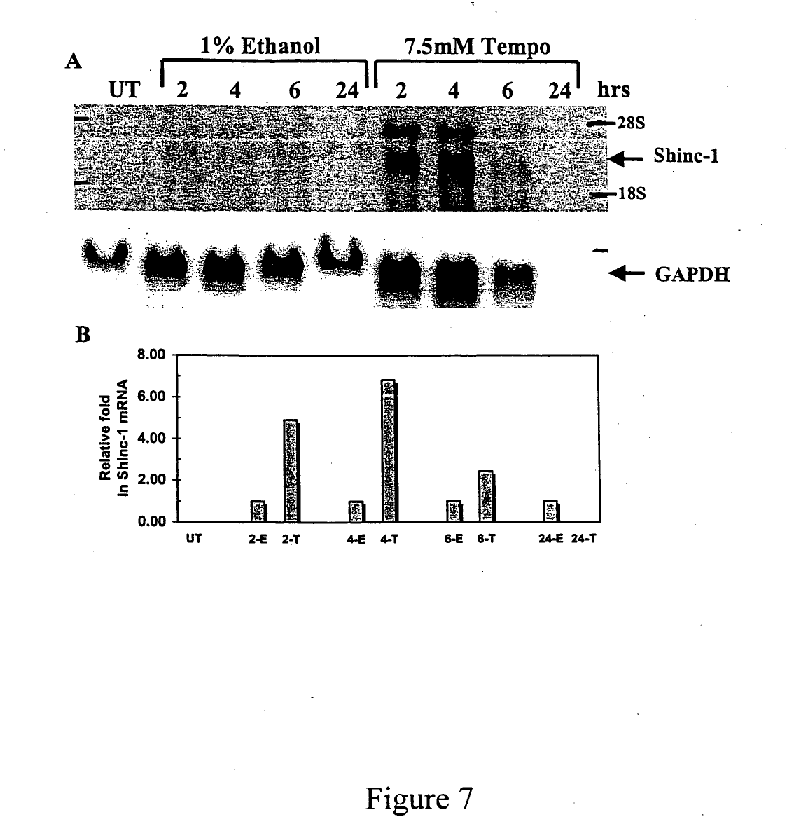

FIG. 7. Time-course analysis of Shinc-1 mRNA expression in tempo-treated cells. (A) DU-145 cells were treated with 7.5 mM tempo or 1% ethanol for the indicated time. Total RNA were extracted from the cells and fractionated by electrophoresis. Hybridizations were carried out with radiolabeled partial Shinc-1 cDNA probe and the radiolabeled GAPDH cDNA probe. (B) Autoradiographs were computer-scanned using the Image-Quant software (Molecular Dynamics). Relative fold change in the steady-state mRNAs level were calculated by normalizing against the GAPDH signal, followed by comparison with the expression in ethanol-treated and tempo-treated cells. UT: untreated, E: ethanol, T: tempo.

### DETAILED DESCRIPTION OF THE INVENTION

The molecular genetic factors that negate cell death and contribute to tumor growth and metastasis can be attractive targets for therapeutic intervention. In a search for such genes, the present inventors have identified a cDNA fragment encoding a gene which is hereby named as SHINC-1 and the expression of which is enhanced through cell exposure to Tempo, an inducer of apoptosis.

It has been reported that nitroxide tempo, a low molecular weight antioxidant, is a novel inducer of apoptosis or programmed cell death in a variety of human cancer cells (Suy et al., JBC, 273: 1781, 1998). Tempo-treatment of tumor-bearing athymic mice causes tumor growth arrest or tumor regression (Suy et al., manuscript in preparation). Members of the mitogen-activated protein kinase (MAPK) family, including ERKs (p42/22 MAPKs), the stress-activated protein kinases (SAPKs) (also called c-Jun NH₂-terminal kinases (p46/52 JNKs/SAPKs)), and p38 MAPK (also termed reactivating kinase (p38RK)), are activated in response to a variety of cellular stresses, such as changes in osmolarity and metabolism, DNA damage, heat shock, ischemia, UV radiation, ionizing radiation, or inflammatory cytokines. In many of these instances, free radicals and derived species play an important role in initiating a cellular signal transduction response. Mechanisms of tempo-induced cell death are unclear, although SAPK and caspases appear to be potential downstream effectors in this pathway. In this study, we used differential display of mRNA strategy to compare the gene expression profiles of tempo-treated (7.5 mM, 2 h) and control (1% ethanol, 2 h; untreated) cancer cells. A novel partial cDNA fragment (485 bp, Shinc-1) was isolated. Two mRNA transcripts of Shinc-1 gene, ˜2.8 kb and ˜5.0 kb, were identified in most normal human tissues, and some human cancer cell lines. Tempo-treatment of breast and prostate cancer cells led to an elevation in the steady state mRNA level of Shinc-1 (DU-145, ˜1.8-fold; MDA-MB231, ˜1.5-fold).

The expression of the gene SHINC-1 (FIGS. 5 and 6) is increased in human cancer cells (treated with Tempo. Tempo causes programmed cell death in cancer cells. Thus, the present inventors have shown that SHINC-1 is involved in growth and cell survival/death pathway in cancer cells and is regulated by Tempo or Tempo promoted cycle components.

In identifying the role played SHINC-1 the inventors have examined changes in gene expression profiles in numerous cancer cells treated with Tempo (FIGS. 5 and 6).

Gene expression profiles of tempo-treated (7.5 mM, 2 h) and control (1% ethanol, 2 h; untreated) cells were compared. The partial cDNA fragments of four differentially expressed mRNAs were subcloned and sequenced. Three of these fragments were identified as human heat shock protein 105 α (526 bp, HSP105α), mitochondrial NADH dehydrogenase subunit 2 (228 bp, ND2), and mitochondrial NADH dehydrogenase subunit 3 (285 bp, ND3). The fourth fragment was found to be a novel cDNA (485 bp, Shinc-1). Northern blot analysis indicated an increase in the steady state mRNA level of HSP105α in PC-3 (˜3.5-fold) and MDA-MB 231 cells (˜5.0-fold) treated with tempo as compared with vehicle-treated cells. ND2 and ND3 mRNA levels were decreased in tempo-treated cells (ND2: PC-3, ˜2.0-fold, DU-145, ˜1.7-fold, MDA-MB 231, ˜1.7-fold; ND3: PC-3, ˜2.8-fold, MDA-MB 231, ˜2.0-fold). Two transcripts of Shinc-1, ˜2.8 kb and ˜5.0 kb, were identified in most normal human tissues, and tempo-treatment of cells led to an elevation in the steady state mRNA level of Shinc-1 (DU-145, ˜1.8-fold; MDA-MB 231,˜1.50-fold). HSP105α, ND2 and ND3 have been previously implicated in stress response andenergy metabolism.

Based on these discoveries, the present invention relates to a novel gene, SHINC-1, the expression of which is increased by agents which mediate apoptosis, the corresponding polypeptide, and application thereof in diagnostic and therapeutic methods. Particularly, the invention provides a novel target for identifying compounds that promote apoptosis of cancer cells, especially breast and lung cancer.

As noted, the invention is broadly directed to a novel gene referred to as SHINC-1. Reference to SHINC-1 herein is intended to be construed to include SHINC-1 proteins of any origin which are substantially homologous to and which are biologically equivalent to the SHINC-1 characterized and described herein. Such substantially homologous SHINC-1 may be native to any tissue or species and, similarly, biological activity can be characterized in any of a number of biological assay systems.

The term “biologically equivalent” is intended to mean that the compositions of the present invention are capable of demonstrating some or all of the same biological properties in a similar fashion, not necessarily to the same degree as the SHINC-1 isolated as described herein or recombinantly produced human SHINC-1 of the invention.

By “substantially homologous” it is meant that the degree of homology of human SHINC-1 from any species is greater than that between SHINC-1 and any previously reported apoptopic modulating gene.

Sequence identity or percent identity is intended to mean the percentage of same residues between two sequences, wherein the two sequences are aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=I0; gap length penalty for multiple alignment=10; k-tuple value in pairwise alignment=1; gap penalty in pairwise alignment=3; window value in pairwise alignment=5; diagonals saved in pairwise alignmentz=5. The residue weight table used for the alignment program is PAM25O (Dayhoffet al., in Atlas of Protein Sequence and Structure, Dayhoff, Ed., NDRF, Washington, Vol. 5, suppl. 3, p. 345, 1978).

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human SHINC-1 when determining percent conservation with non-human SHINC-1, and referenced to SHINC-1 when determining percent conservation with non-SHINC-1 proteins. Conservative amino acid changes satisfying this requirement are: R-K; E-D, Y-F, L-M; V-I, Q-H.

Polypeptide Fragments

The invention provides polypeptide fragments of the disclosed proteins. Polypeptide fragments of the invention can comprise at least 8, 10, 12, 15, 18, 19, 20, 25, 50, 75, 100, or 108 contiguous amino acids of an amino acid sequence encoded by a nucleic acid sequence comprising the sequence contained in FIG. 1 (SEQ ID NO 1). Also included are all intermediate length fragments in this range, such as 51, 52, 53, etc.; 70, 71, 72, etc.; and 100, 101, 102, etc., which are exemplary only and not limiting.

Biologically Active Variants

Variants of the SHINC-1 polypeptide disclosed herein can also occur. Variants can be naturally or non-naturally occurring. Naturally occurring variants are found in humans or other species and comprise amino acid sequences which are substantially identical to the amino acid sequence shown in FIG. 1 (SEQ ID NO 1). Species homologs of the protein can be obtained using subgenomic polynucleotides of the invention, as described below, to make suitable probes or primers to screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants are also included here. Preferably, naturally or non-naturally occurring variants have amino acid sequences which are at least 85%, 90%, or 95% identical to the amino acid sequence encoded by a nucleic acid sequence comprising the sequence shown in FIG. 1 (SEQ ID NO 1). More preferably, the molecules are at least 96%, 97%, 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. (1981) 2:482-489.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of SHINC-1 or polypeptide variants are of the same type as a protein comprising the amino acid sequence encoded by a nucleic acid sequence comprising the nucleotide sequence shown in FIG. 1 (SEQ ID NO 1), although the properties and functions of variants can differ in degree.

SHINC-1 protein variants include glycosylated forms, aggregative conjugates with other molecules, and covalent conjugates with unrelated chemical moieties. SHINC-1 protein variants also include allelic variants, species variants, and muteins. Truncations or deletions of regions which do not affect the differential expression of the SHINC-1 protein gene are also variants. Covalent variants can be prepared by linking functionalities to groups which are found in the amino acid chain or at the N- or C-terminal residue, as is known in the art.

It will be recognized in the art that some amino acid sequence of the SHINC-1 protein of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the SHINC-1 polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244:108 1-1085 (1989)). The latter procedure introduces single alanine mutations at every residue in the molecule. The resulting mutant molecules are then tested for biological activity such as binding to a natural or synthetic-binding partner. Sites that are critical for ligand-receptor binding can also be determined by structural analysis such as crystallization, nuclear magnetic resonance or photoaffinity labeling (Smith et al., J Mol. Biol. 224:899-904 (1992) and de Vos et al. Science 255:306-3 12 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Fusion Proteins

Fusion proteins comprising proteins or polypeptide fragments of SHINC-1 can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of SHINC-1 or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize an amino acid sequence encoded by a nucleic acid sequence comprising the sequence shown in FIG. 1 (SEQ ID NO 1) or can be prepared from biologically active variants such as those described above. The first protein segment can consist of a full-length SHINC-1 or a portion thereof.

Other first protein segments can consist of at least 8, 10, 12, 15, 18, 19, 20, 25, 50, 75, 100, 108 contiguous amino acids selected from the sequence encoded by the nucleic acid sequence shown in FIG. 1 SEQ ID NO 1. The contiguous amino acids listed herein are not limiting and also include all intermediate lengths such as 20, 21, 22, etc.; 70, 71, 72, etc.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence comprising the sequence contained in FIG. 1 (SEQ ID NO 1) in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a coding sequence of the nucleotide sequence comprising the sequence shown in FIG. 1 (SEQ ID NO 1) can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

SHINC-1 protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the sequence contained in FIG. 1 (SEQ ID NO 1). Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence shown in FIG. 1 (SEQ ID NO 1) for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Host Cells

An expression construct can be introduced into a host cell. The host cell comprising the expression construct can be any suitable prokaryotic or eukaryotic cell. Expression systems in bacteria include those described in Chang et al., Nature (1978) 275:615; Goeddel et al., Nature (1979) 281: 544; Goeddel et al., Nucleic Acids Res. (1980) 8:4057; EP 36,776; U.S. Pat. No. 4,551,433; deBoer et al., Proc. Natl. Acad Sci. USA (1983) 80: 21-25; and Siebenlist et al., Cell (1980) 20: 269.

Expression systems in yeast include those described in Hinnen et al., Proc. Natl. Acad. Sci. USA (1978) 75: 1929; Ito et al., J Bacteriol. (1983) 153: 163; Kurtz et al., Mol. Cell. Biol. (1986) 6:142; Kunze et al., J Basic Microbiol. (1985) 25: 141; Gleeson et al., J. Gen. Microbiol. (1986) 132: 3459, Roggenkamp et al., Mol. Gen. Genet. (1986) 202:302); Das et al., J Bacteriol. (1984) 158: 1165; De Louvencourt et al., J Bacteriol. (1983) 154: 737, Van den Berg et al., Bio/Technology (1990) 8: 135; Kunze et al., J. Basic Microbiol. (1985) 25: 141; Cregg et al., Mol. Cell. Biol. (1985) 5: 3376; U.S. Pat. No. 4,837,148; U.S. Pat. No. 4,929,555; Beach and Nurse, Nature (1981) 300: 706; Davidow et al., Curr. Genet. (1985) 1p: 380; Gaillardin et al., Curr. Genet. (1985) 10: 49; Ballance et al., Biochem. Biophys. Res. Commun. (1983) 112: 284-289; Tilburn et al., Gene (1983) 26: 205-22; Yelton et al., Proc. Natl. Acad, Sci. USA (1984) 81: 1470-1474; Kelly and Hynes, EMBO J. (1985) 4: 475479; EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. (1988) 69: 765-776; Miller et al., Ann. Rev. Microbiol. (1988) 42: 177; Carbonell et al., Gene (1988) 73: 409; Maeda et al., Nature (1985) 315: 592-594; Lebacq-Verheyden et al., Mol. Cell Biol. (1988) 8: 3129; Smith et al., Proc. Natl. Acad. Sci. USA (1985) 82: 8404; Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) 6: 47-55, Miller et al., in GENERIC ENGINEERING (Setlow, J. K. et al. eds.), Vol. 8 (Plenum-Publishing, 1986), pp. 277-279; and Maeda et al., Nature, (1985) 315: 592-594.

Mammalian expression can be accomplished as described in Dijkema et al., EMBO J. (1985) 4: 761; Gormanetal., Proc. Natl. Acad. Sci. USA (1982b) 79: 6777; Boshart et al., Cell (1985) 41: 521; and U.S. Pat. No. 4,399,216. Other features of mammalian expression can be facilitated as described in Ham and Wallace, Meth Enz. (1979) 58: 44; Barnes and Sato, Anal. Biochem. (1980) 102: 255; U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides from the nucleotide sequence shown in FIG. 1 (SEQ ID NO 1). The transcription unit is located upstream to a coding sequence of the endogenous gene. The exogenous regulatory sequence directs transcription of the coding sequence of the endogenous gene.

SHINC-1 can also include hybrid and modified forms of SHINC-1 proteins including fusion proteins, SHINC-1 fragments and hybrid and modified forms in which certain amino acids have been deleted or replaced, modifications such as where one or more amino acids have been changed to a modified amino acid or unusual amino acid, and modifications such as glycosylations so long as the hybrid or modified form retains at least one of the biological activities of SHINC-1. By retaining the biological activity of SHINC-1, it is meant that the protein modulates cancer cell proliferation or apoptosis, although not necessarily at the same level of potency as that of SHINC-1 as described herein.

Also included within the meaning of substantially homologous is any SHINC-1 which may be isolated by virtue of cross-reactivity with antibodies to the SHINC-1 described herein or whose encoding nucleotide sequences including genomic DNA, mRNA or cDNA may be isolated through hybridization with the complementary sequence of genomic or subgenomic nucleotide sequences or cDNA of the SHINC-1 herein or fragments thereof. It will also be appreciated by one skilled in the art that degenerate DNA sequences can encode human SHINC-1 and these are also intended to be included within the present invention as are allelic variants of SHINC-1.

Preferred SHINC-1 of the present invention have been identified and isolated in purified form as described, Also preferred is SHINC-1 prepared by recombinant DNA technology. By “pure form” or “purified form” or “substantially purified form” it is meant that a SHINC-1 composition is substantially free of other proteins which are not SHINC-1.

The present invention also includes therapeutic or pharmaceutical compositions comprising SHINC-1 in an effective amount for treating patients with disease, and a method comprising administering a therapeutically effective amount of SHINC-1. These compositions and methods are useful for treating a number of diseases including cancer. One skilled in the art can readily use a variety of assays known in the art to determine whether SHINC-1 would be useful in promoting survival or functioning in a particular cell type.

In certain circumstances, it may be desirable to modulate or decrease the amount of SHINC-1 expressed. Thus, in another aspect of the present invention, SHINC-1 anti-sense oligonucleotides can be made and a method utilized for diminishing the level of expression of SHINC-1 by a cell comprising administering one or more SHINC-1 anti-sense oligonucleotides. By SHINC-1 anti-sense oligonucleotides reference is made to oligonucleotides that have a nucleotide sequence that interacts through base pairing with a specific complementary nucleic acid sequence involved in the expression of SHINC-1 such that the expression of SHINC-1 is reduced. Preferably, the specific nucleic acid sequence involved in the expression of SHINC-1 is a genomic DNA molecule or mRNA molecule that encodes SHINC-1. This genomic DNA molecule can comprise regulatory regions of the SHINC-1 gene, or the coding sequence for mature SHINC-1 protein.

The term complementary to a nucleotide sequence in the context of SHINC-1 antisense oligonucleotides and methods therefor means-sufficiently complementary to such a sequence as to allow hybridization to that sequence in a cell, i.e., under physiological conditions. The SHINC-1 antisense oligonucleotides preferably comprise a sequence containing from about 8 to about 100 nucleotides and more preferably the SHINC-1 antisense oligonucleotides comprise from about 15 to about 30 nucleotides. The SHINC-1 antisense oligonucleotides can also contain a variety of modifications that confer resistance to nucleolytic degradation such as, for example, modified internucleoside linages (Uhlmann and Peyman, Chemical Reviews 90:543-548 1990; Schneider and Banner, Tetrahedron Lett. 31:335, 1990 which are incorporated by reference), modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art, a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth, proliferation or viability as is known in the art. Assays for measuring apoptosis are also known.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.)

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

SHINC-1 can also be linked or conjugated with agents that provide desirable pharmaceutical or pharmacodynamic properties. For example, SHINC-1 can be coupled to any substance known in the art to promote penetration or transport across the blood-brain barrier such as an antibody to the transferrin receptor, and administered by intravenous injection. (see, for example, Friden et al., Science 259:373-377, 1993 which is incorporated by reference). Furthermore, SHINC-1 can be stably linked to a polymer such as polyethylene glycol to obtain desirable properties of solubility, stability, half-life and other pharmaceutically advantageous properties. (See, for example, Davis et al., Enzyme Eng. 4:169-73, 1978; Buruham, Am. J. Hosp. Pharm. 51:210-218,1994 which are incorporated by reference.)

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. SHINC-1 can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing SHINC-1 are to be administered orally. Such formulations are preferably encapsulated and formulated with suitable carriers in solid dosage forms. Some examples of suitable carriers, excipients, and dilutents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, SHINC-1 may be therapeutically administered by implanting into patients vectors or cells capable of producing a biologically-active form of SHINC-1 or a precursor of SHINC-1, i.e., a molecule that can be readily converted to a biological-active form of SHINC-1 by the body. In one approach cells that secrete SHINC-1 may be encapsulated into semipermeable membranes for implantation into a patient. The cells can be cells that normally express SHINC-1 or a precursor thereof or the cells can be transformed to express SHINC-1 or a precursor thereof. It is preferred that the cell be of human origin and that the SHINC-1 be human SHINC-1 when the patient is human. However, the formulations and methods herein can be used for veterinary as well as human applications and the term “patient” as used herein is intended to include human and veterinary patients.

In a number of circumstances it would be desirable to determine the levels of SHINC-1 in a patient. The identification of SHINC-1 along with the present report showing expression of SHINC-1 provides the basis for the conclusion that the presence of SHINC-1 serves a normal physiological function related to cell growth and survival. Endogenously produced SHINC-1 may also play a role in certain disease conditions.

The term “detection” as used herein in the context of detecting the presence of SHINC-1 in a patient is intended to include the determining of the amount of SHINC-1 or the ability to express an amount of SHINC-1 in a patient, the estimation of prognosis in terms of probable outcome of a disease and prospect for recovery, the monitoring of the SHINC-1 levels over a period of time as a measure of status of the condition, and the monitoring of SHINC-1 levels for determining a preferred therapeutic regimen for the patient.

To detect the presence of SHINC-1 in a patient, a sample is obtained from the patient. The sample can be a tissue biopsy sample or a sample of blood, plasma, serum, CSF or the like. SHINC-1 tissue expression is disclosed in the examples. Samples for detecting SHINC-1 can be taken from these tissue. When assessing peripheral levels of SHINC-1, it is preferred that the sample be a sample of blood, plasma or serum. When assessing the levels of SHINC-1 in the central nervous system a preferred sample is a sample obtained from cerebrospinal fluid or neural tissue.

In some instances it is desirable to determine whether the SHINC-1 gene is intact in the patient or in a tissue or cell line within the patient. By an intact SHINC-1 gene, it is meant that there are no alterations in the gene such as point mutations, deletions, insertions, chromosomal breakage, chromosomal rearrangements and the like wherein such alteration might alter production of SHINC-1 or alter its biological activity, stability or the like to lead to disease processes. Thus, in one embodiment of the present invention a method is provided for detecting and characterizing any alterations in the SHINC-1 gene. The method comprises providing an oligonucleotide that contains the SHINC-1 cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize to the SHINC-1 gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, Taql and Alul. Using the Southern blot protocol, which is well known in the art, this assay determines whether a patient or a particular tissue in a patient has an intact SHINC-1 gene or a SHINC-1 gene abnormality.

Hybridization to a SHINC-1 gene would involve denaturing the chromosomal DNA to obtain a single-stranded DNA; contacting the single-stranded DNA with a gene probe associated with the SHINC-1 gene sequence; and identifying the hybridized DNA-probe to detect chromosomal DNA containing at least a portion of a human SHINC-1 gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The SHINC-1 gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

SHINC-1 gene abnormalities can also be detected by using the PCR method and primers that flank or lie within the SHINC-1 gene. The PCR method is well known in the art. Briefly, this method is performed using two oligonucleotide primers which are capable of hybridizing to the nucleic acid sequences flanking a target sequence that lies within a SHINC-1 gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising SHINC-1 or a fragment thereof is then directly sequenced and analyzed by comparison of the sequence with the sequences disclosed herein to identify alterations which might change activity or expression levels or the like.

In another embodiment, a method for detecting SHINC-1 is provided based upon an analysis of tissue expressing the SHINC-1 gene. Certain tissues such as those identified below in Example 6 and 7 have been found to express the SHINC-1 gene. The method comprises hybridizing a polynucleotide to mRNA from a sample of tissue that normally expresses the SHINC-1 gene. The sample is obtained from a patient suspected of having an abnormality in the SHINC-1 gene or in the SHINC-1 gene of particular cells.

To detect the presence of mRNA encoding SHINC-1 protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding SHINC-1 protein or a derivative of the cDNA as a probe, high stringency conditions can be used in order to prevent false positives, that is the hybridization and apparent detection of SHINC-1 nucleotide sequences when in fact an intact and functioning SHINC-1 gene is not present. When using sequences derived from the SHINC-1 cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. (Sambrook et al., 1989, supra).

In order to increase the sensitivity of the detection in a sample of mRNA encoding the SHINC-1 protein, the technique of reverse transcription/polymerization chain reaction (RT/PCR) can be used to amplify cDNA transcribed from mRNA encoding the SHINC-1 protein. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and SHINC-1 specific primers. (Belyavsky et al., Nucl. Acid Res. 17:2919-2932, 1989; Krug and Berger, Methods in Enzymology, 152:316-325, Academic Press, NY, 1987 which are incorporated by reference).

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the SHINC-1 protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. (Basic and Clinical Immunology, 217-262, Sites and Terr, eds., Appleton & Lange, Norwalk, Conn., 1991, which is incorporated by reference). Preferred are binder-ligand immunoassay methods including reacting antibodies with an epitope or epitopes of the SHINC-1 protein and competitively displacing a labeled SHINC-1 protein or derivative thereof.

As used herein, a derivative of the SHINC-1 protein is intended to include a polypeptide in which certain amino acids have been deleted or replaced or changed to modified or unusual amino acids wherein the SHINC-1 derivative is biologically equivalent to SHINC-1 and wherein the polypeptide derivative cross-reacts with antibodies raised against the SHINC-1 protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, usually a rabbit or a mouse.

Oligopeptides can be selected as candidates for the production of an antibody to the SHINC-1 protein based upon the oligopeptides lying in hydrophilic regions, which are thus likely to be exposed in the mature protein. Peptide sequence used to generate antibodies against any fragment of SHINC-1 that typically is at least 5-6 amino acids in length, optionally fused to an immunogenic carrier protein, e.g. KLH or BSA.

Additional oligopeptides can be determined using, for example, the Antigenicity Index, Welling, G. W. et al., FEBS Lett. 188:215-218 (1985), incorporated herein by reference.

In other embodiments of the present invention, humanized monoclonal antibodies are provided, wherein the antibodies are specific for SHINC-1. The phrase “humanized antibody” refers to an antibody derived from a non-human antibody, typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking”. them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci, USA, 81:6851-6855 (1984); Morrison and Oi, Adv. Immunol., 44:65-92 (1988); Verhoeyer et al., Science 239:1534-1536 (1.988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which patents are incorporated herein by reference.

Humanized antibodies to SHINC-1 can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNF, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, SHINC-1 polypeptides of the invention and variants thereof are used to immunize a transgenic animal as described above. Monoclonal antibodies are made using methods known in the art, and the specificity of the antibodies is tested using isolated SHINC-1 polypeptides.

Methods for preparation of the SHINC-1 protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E. I. du Pont de Nemours Company, Wilmington, Del.) (Caprino and Han, J. Org. Chem. 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified SHINC-1 protein usually by ELISA or by bioassay based upon the ability to block the action of SHINC-1. In a non-limiting example, an antibody to SHINC-1 can block the binding of SHINC-1 to Disheveled protein. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the SHINC-1 protein by treatment of a patient with specific antibodies to the SHINC-1 protein.

Specific antibodies, either polyclonal or monoclonal, to the SHINC-1 protein can be produced by any suitable method known in the art as discussed above. For example, murine or human monoclonal antibodies can be produced by hybridoma technology or, alternatively, the SHINC-1 protein, or an immunologically active fragment thereof, or an anti-idiotypic antibody, or fragment thereof can be administered to an animal to elicit the production of antibodies capable of recognizing and binding to the SHINC-1 protein. Such antibodies can be from any class of antibodies including, but not limited to IgG, IgA, 1gM, IgD, and IgE or in the case of avian species, IgY and from any subclass of antibodies.

The availability of SHINC-1 allows for the identification of small molecules and low molecular weight compounds that inhibit the binding of SHINC-1 to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbence, fluorescence or luminescence as read-outs. Gonzalez, J. E. et al., (1998) Curr. Opin. Biotech. 9:624-63 1.

Model systems are available that can be adapted for use in high throughput screening for compounds that inhibit the interaction of SHINC-1 with its ligand, for example by competing with SHINC-1 for ligand binding. Sarubbi et al., (1996) Anal. Biochem. 237:70-75 describe cell-free, non-isotopic assays for discovering molecules that compete with natural ligands for binding to the active site of IL-1 receptor. Martens, C. et al., (1999) Anal. Biochem. 273:20-31 describe a generic particle-based nonradioactive method in which a labeled ligand binds to its receptor immobilized on a particle; label on the particle decreases in the presence of a molecule that competes with the labeled ligand for receptor binding.

The therapeutic SHINC-1 polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplift, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/0793 6; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/3 0763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., P.N.A.S. 90:10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisleret al., P.N.A.S. 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO. 95/00655. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone, for example Curiel, Hum. Gene Ther. 3:147-154 (1992); ligand-linked DNA, for example see Wu, J. Biol. Chem. 264:16985-16987 (1989); eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13 796, WO 94/23697, and WO 9 1/14445, and EP No. 0 524 968.

Further non-viral delivery suitable for use includes mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

SHINC-1 may also be used in screens to identify drugs for treatment of cancers which involve over-activity of the encoded protein, or new targets which would be useful in the identification of new drugs.

For all of the preceding embodiments, the clinician will determine, based on the specific condition, whether SHINC-1 polypeptides or polynucleotides, antibodies to SHINC-1, or small molecules such as peptide analogues or antagonists, will be the most suitable form of treatment. These forms are all within the scope of the invention.

### EXAMPLE

Cell culture—-DU145 human prostate cancer cells, PC-3 human prostate cancer cells and MDA-MB231 human breast cancer cells were grown in improved minimum essential medium (Cellgro) containing 10% fetal bovine serum and 2 mM L-glutamine in a humidified atmosphere of 5% CO2: 95% air at 37° C.

Reamplification and Cloning of cDNA Fragments—Bands of interest corresponding to mRNAs upregulated or downregulated in the tempo-treated cells as compared to 1% ethanol treated and untreated cells or the converse, were located on the differential display gel and excised, and DNA was eluted by soaking the bands in 100 μl of H2O for 10 min and then boiling for 15 min. The supernatant was precipitated with ethanol and then sample was dissolved in 10 μl of H2O and reamplified using the original combination of the arbitrary and anchor primers according to the instructions in the RNAimage kit. If the amplified product was not detectable by 1.5% agarose gel electrophoresis, a third-step PCR as described above was carried out using a 1:10 dilution of the reamplified PCR product. The PCR product was cloned into the PCR 2.1 cloning vector according to the TA cloning kit instructions (Invitrogen, San Diego, Calif.). Plasmid DNA isolation from overnight cultures of the transformed E.coli cells (One Shot, INV αF′; Invitorgen) was carried out by the alkaline lysis and phenol/chloroform extraction method. Size of the insert cDNA was determined by restriction digestion with EcoR1, followed by agarose gel electrophoresis. Inserts of expected sizes were purified from the agarose gel according to the Qiax 2 kit (Qiagen, Chatsworth, Calif.).

cDNA Sequencing—The partial cDNA clones representing differentially expressed mRNAs were sequenced in both directions, using either the T7 or M13 reverse primer (Perkin Elmer) by the automated DNA sequencer (Applied Biosystems, Perkin Elmer). The cDNA sequences were subsequently entered in the DNA databases (GenBank, DDBJ, GenEMBL, Human EST) to examine the homology to the known genes.

Northern Blot Hybridization Analysis—Total RNA extracted from the cells was fractionated on a 1.0% formaldehyde agarose gel and transferred onto nylon membrane (Qiagen) and fixed by UV cross-linking. cDNA inserts and GAPDH cDNA probe were radiolabeled with ³²P-dCTP using a random primer DNA labeling kit (Pharmacia Biotech, Piscataway, N.J.). Blots were sequentially hybridized first to a radiolabeled partial human cDNA probe and then to GAPDH cDNA probe at 68° C. in ExpressHyb Hybridization Solution (Clontech, Palo Alto, Calif.). Blots were washed three times in 2×SSC and 0.05% SDS at 68° C., 2 times in 0.1×SSC and 0.1% SDS at 50° C. Dried blots were exposed to X-ray films at −80° C. Autoradiographs were computer-scanned using the Image-Quant software, version 5.1 (Molecular Dynamics, Sunnyvale, Calif.). Expression of Shinc-1 cDNA fragment was also examined on 2 μg per lane poly (A)⁺ mRNA blots of multiple human tissue and human cancer cell lines (Clontech). These blots were sequentially hybridized with β-actin cDNA probe according to the manufacturer's instructions.

Isolation of a partial Shinc-1 cDNA fragment—We compared the patterns of differentially displayed mRNAs in untreated, 1% ethanol-treated cells and tempo-treated DU-145 prostate cancer cells (7.5 mM, 2 h). Twenty-four different anchor and arbitrary primer combinations were tested to identify differentially displayed mRNAs in these three categories. The overall patterns of the amplified cDNA species were essentially the same and any specific differences within the cells were easily visually identifiable. Each primer combination displayed approximately 150-200 bands, each band theoretically representing one transcribed gene. The selection of a differentially expressed mRNA band was based on the presence of this band in untreated and ethanol treated cells and its absence in tempo-treated cells or the converse, i.e., the presence of a band in tempo-treated cells and its absence in the untreated and ethanol-treated cells. If a band was present in all categories, selection was based on a significant visual difference in the band intensity noted in these cells. A fragment C4-1U was selected on the basis of the relatively higher band intensity in tempo-treated DU-145 cells. The selected fragment was eluted from the gel, purified, and reamplified. The fragment exhibited a single band upon reamplification (data not shown). The cDNA fragment was then cloned into the TA cloning vector.

Nucleotide sequencing analysis and DNA database homology search of the partial cDNA fragments were performed. The partial sequence cDNA sequences of C4-1U (485 bp) showed no significant homology to any of the sequences in the Genbank, DDBJ and GenEMBL database or human EST database, indicating that this cDNA represents the novel gene (FIG. 1).

Expression of SHINC-1, Novel Gene, in Human Normal Tissues and Cancer Cell Lines—Two transcripts of Shinc-1 (˜2.8 kb, ˜5.0 kb) were detected in most normal human tissues and several human cancer cell types by northern blot analysis using normal tissues blots and cancer cell line blot (FIGS. 2-4). Shinc-1 is expressed in all human tissues and most of human cancer cell types examined. Both transcripts were relatively less in brain and thymus.

Expression of Shinc-1 in tempo-treated prostate (DU-145, PC-3) and breast cancer cells (MDA-MB231)—Northern blot analysis was performed with radiolabeled partial cDNA insert of Shinc-1. A major Shinc-1 transcript approximately 2.8 kb was detected in cancer cells and Shinc-1 mRNA level was increased in tempo-treated cells (DU-145, ˜1.8-fold; MDA-MB231, ˜1.5-fold) (FIGS. 5 and 6). We next examined level of the Shinc-1 mRNA through time-course of tempo treatment. Shinc-1 mRNA increased at 2 h-4 h post-tempo treatment of DU-145 prostate cancer cells (FIG. 7). The results show that expression of Shinc-1 gene was regulated by tempo treatment in a time-dependent manner.

The present invention has been described with reference to specific embodiments. However, this invention is intended to cover those changes and substitutions, which may be made by those skilled in the art without departing from the spirit and scope of the appended claims.

What is claimed is:

1. An isolated nucleic acid molecule comprising a polynucleotide selected from the group consisting of:
(a) a polynucleotide comprising nucleotides 1 to about 456 of the nucleic acid sequence contained in SEQ ID NO:1;
(b) a polynucleotide comprising nucleotides 2 to about 456 of the nucleic acid sequence contained in SEQ ID NO:1;
(c) the polynucleotide complement of the polynucleotide of (a) or (b); and
(d) a polynucleotide at least 90% identical to the polynucleotide of (a), (b) or (c).

2. An isolated nucleic acid molecule comprising about 10 to about 456 contiguous nucleotides from the nucleic acid sequence contained in SEQ ID NO1.

3. An isolated nucleic acid molecule comprising about 50 to about 200 contiguous nucleotides from the nucleic acid sequence contained in SEQ ID NO:1.

4. An isolated nucleic acid molecule comprising about 100 to about 400 contiguous nucleotides of the nucleic acid sequence contained in SEQ ID NO:1.

5. An isolated nucleic acid molecule comprising about 10 to about 300 contiguous nucleotides from the nucleic acid sequence contained in SEQ ID NO:1.

6. An isolated nucleic acid molecule comprising about 100 to about 300 contiguous nucleotides from the nucleic acid sequence contained in SEQ ID NO:1.

7. The isolated nucleic acid molecule of claim 1, which is DNA.

8. A method of making a recombinant vector comprising inserting a nucleic acid molecule of claim 1 into a vector in operable linkage to a promoter.

9. A recombinant vector produced by the method of claim 8.

10. A method of making a recombinant host cell comprising introducing the recombinant vector of claim 9 into a host cell.

11. A recombinant host cell produced by the method of claim 10.

12. A recombinant method of producing a polypeptide, comprising culturing the recombinant host cell of claim 11 under conditions such that said polypeptide is expressed and recovering said polypeptide.

13. An isolated polypeptide comprising amino acids at least 95% identical to amino acids encoded by at least 100 contiguous nucleic acids from the sequence contained in SEQ ID NO:1.

14. An isolated polypeptide comprising amino acids at least 95% identical to amino acids encoded by at least 200 contiguous nucleic acids from the sequence contained in SEQ ID NO:1.

15. An isolated polypeptide comprising amino acids at least 95% identical to amino acids encoded by at least 300 contiguous nucleic acids from the sequence contained in SEQ ID NO:1.

16. An epitope-bearing portion of the polynucleotide encoded by a nucleic acid sequence comprising the sequence identified in SEQ ID NO:1.

17. The epitope-bearing portion of claim 16, which comprises about 5 to about 30 amino acids encoded by contiguous nucleic acids from a sequence comprising the sequence identified in SEQ ID NO: 1.

18. The epitope-bearing portion of claim 17, which comprises about 10 to about 15 amino acids encoded by contiguous nucleic acids from a sequence comprising the sequence of SEQ ID NO:1.

19. An isolated antibody that binds specifically to the polypeptide of claim 15.

20. A monoclonal antibody according to claim 19.

21. A method of modulating apoptosis or proliferation of a cancer cell, comprising regulating expression of SHINC-1 in said mammalian cell.

22. The method of claim 21, wherein said mammalian cell is transformed with a vector encoding an antisense oligonucleotide corresponding to a sequence comprising the sequence of SEQ ID NO:1.

23. An antisense oligonucleotide that inhibits the expression of SHINC-1 in a mammalian cell.

24. The antisense oligonucleotide of claim 23 which is contained in a liposomal formulation.

25. A method of treating cancer characterized by SHINC-1 overexpression by administration of an antisense oligonucleotide or ribozyme that inhibits SHINC-1 expression.

26. A method of treating cancer characterized by SHINC-1 overexpression comprising administering an antibody that specifically binds SHINC-1.

27. A method of detecting cancer characterized by SHINC-1 overexpression or underexpression comprising detecting the levels of SHINC-1 expression and correlating said level of expression to the presence or absence of cancer.

28. The method of claim 27 which is effected by using a cDNA that hybridizes to SHINC-1 mRNA.

29. The method of claim 27 which is effected by]by using an antibody that specifically binds SHINC-1.

30. A method for inhibiting cancer cell proliferation and/or metastasis in a cancer patient comprising administering a ribozyme or antisense oligonucleotide that modulates SHINC-1 expression.

31. The method of claim 30, wherein said cancer is selected from the group consisting of breast cancer, leukemia, lymphoma, melanoma, colorectal cancer, and lung cancer.

32. A method of treating a condition characterized by SHINC-1 underexpression comprising administering an agent that promotes SHINC-1 expression.

33. The method of claim 32, wherein administering an agent that promotes SHINC-1 expression comprises administering SHINC-1 DNA.

34. A method for inhibiting cancer cell proliferation and/or metastasis in a cancer patient comprising administering an antibody that specifically binds SHINC-1; a ribozyme or antisense oligonucleotide that modulates SHINC-1 expression in combination with radio therapy; chemotherapy, hormone or biological anticancer agent.
